(19) United States
(12) Patent Application Publication
(10) Pub. No.: US 2004/0138606 A1
(43) Pub. Date: Jul. 15, 2004

(54) One time use disposable styptic product

(76) Inventors: Kendra D. McPheeters, Las Vegas, NV (US); Phillip A. Cornwell, Umatilla, OR (US); Charles M. Cornwell, Beaverton, OR (US)

Correspondence Address: Kendra D. McPheeters, 4148 Annie Oakley Dr., Las Vegas, NV, 89121, US

(21) Appl. No.: 10/622,289

(22) Filed: Jul. 18, 2003

Related U.S. Application Data
(60) Provisional application No. 60/419,468, filed on Oct. 17, 2002.

(51) Int. Cl. A61M 35/00

(52) U.S. Cl. 604/1

(57) ABSTRACT

The one time use disposable styptic product is an elongated stem-like applicator with an effective amount of styptic product adhered to one or both ends. The length can vary based on the needs of the user. The one time use disposable styptic product remedies several problems never before addressed by manufacturers of the commonly known “Styptic Pencil” or the several powders and gels associated with styptic. Prior styptic applications do not address blood-borne disease control. An one time use, disposable styptic applicator would allow styptic to thrive in industries such as nail salons, barber shops, tattoo and/or piercing parlors and many other similar industries. The product is safely used and disposed of promptly after each use. This is cost effective and a possible solution to the threat of blood-borne diseases in these or similar markets. Styptic is not currently available in a disposable one time use dosage or packaging.

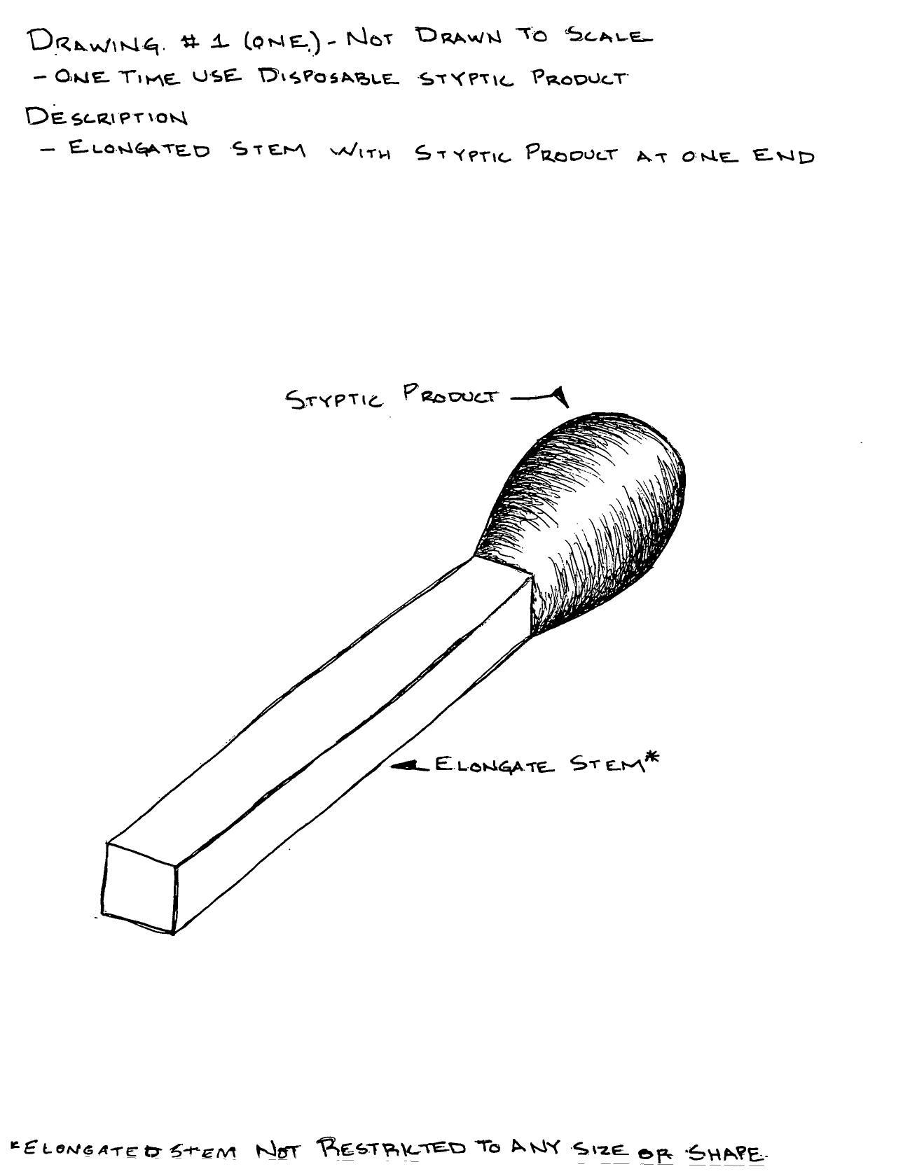

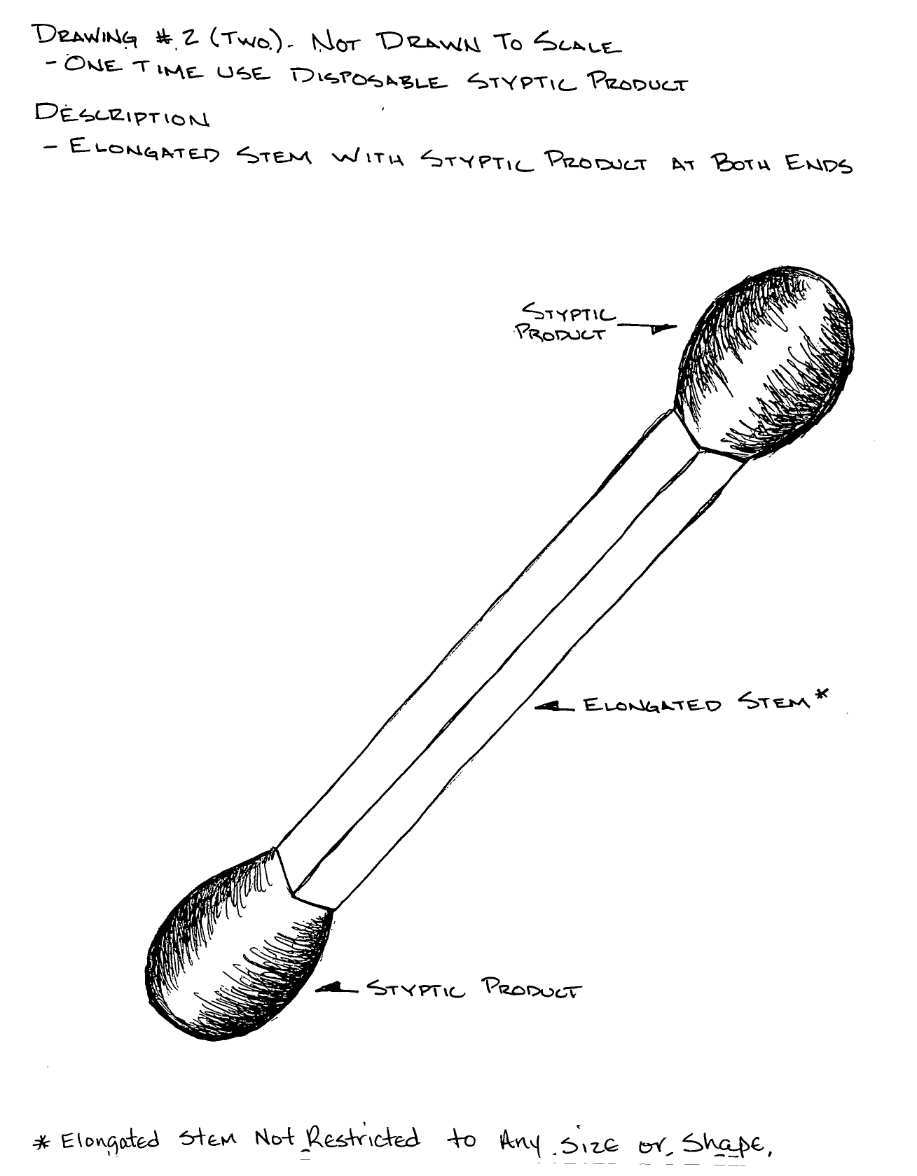

### FIELD OF INVENTION

This invention greatly improves the use of styptic solution by creating an one time use disposable application of styptic solution.

### OBJECT

The objective of the one time use disposable styptic product is to remedy the multiple-use by multiple users problem. We claim an one timely wrapped or package of one time, one-time use disposable styptic applicators be the solution.

### ADVANTAGES

A novelty of the one time use disposable styptic product allows the user to apply a small amount of styptic solution to stop bleeding. A second novelty to our invention is that with a small amount of styptic solution it may be disposed of and another selected when another application is needed without significant loss of product.

### DESCRIPTION OF DRAWINGS

In drawing one (1) the styptic is found on one end of an elongated stem material.

In drawing two (2) the styptic solution is found at both ends of an elongated stem material. The stem length can vary for various uses. The elongated stem of either may be manufactured out of several different man made or natural materials to function as the applicator for this product.

### CONCLUSION

Accordingly the reader will see that the one time use disposable styptic solution applicator permits cost effective use of styptic in the professional or personal use areas. It also provides protection from the risk of the spread of blood-borne diseases. Further objects and advantages of our invention will become apparent from a consideration of the included drawing and description.

1. An one time use disposable styptic applicator comprised of an effective amount of styptic solution adhered to one or both ends of an elongated stem-like applicator.
